(19) United States
(12) Patent Application Publication
(10) Pub. No.: US 2021/0022996 A1
(43) Pub. Date: Jan. 28, 2021

(54) Reduced Friction Eartips, And Systems And Methods Therefore

(71) Applicant: Staton Techiya LLC, Delray Beach, FL (US)

(72) Inventor: John Patrick Keady, Fairfax Station, VA (US)

(73) Assignee: Staton Techiya LLC, Delray Beach, FL (US)

(21) Appl. No.: 16/899,559

(22) Filed: Jun. 11, 2020

Related U.S. Application Data
(60) Provisional application No. 62/879,400, filed on Jul. 26, 2019.

(51) Int. Cl. A61K 9/00; A61F 11/08; B29C 39/00; B29C 39/02; H04R 1/10

(52) U.S. Cl. CPC A61K 9/0046; A61F 11/08; B29C 39/003; B29L 2031/3431; H04R 1/1016; H04R 2201/105; B29C 39/026

(57) ABSTRACT

The application discloses an a method of forming a lower friction eartip when compared to a pretreated elastomer eartip.

### CROSS REFERENCE TO RELATED APPLICATIONS AND PRIORITY

The present application claims priority to and the benefit of U.S. Provisional Patent Application No. 62/879,400, filed Jul. 26, 2019, the entirety of which is hereby incorporated by reference.

### FIELD OF THE APPLICATION

The present application relates to eartips and earplugs with reduced friction, and more particularly, though not exclusively, eartips that have been lubricant treated or coated.

### BACKGROUND

Many devices have been developed over time to deliver acoustic content to a user. Many of these devices take the form of an earphone (a device to deliver audio content directly to the ear, e.g., muff, earbud, in-ear system, hearing aid), which can be connected either wired or wireless to a computational device which delivers content or standalone (e.g., hearing aid). Most designs of earphones have electrical components that are uniquely oriented for a particular form factor and not easily transferrable to other form factors. In essence the electronics are often forced into a form factor rather than a smallest electrical package developed and the form factor developed around the package. Additionally, when the earphone has a component that is at least partially inserted into the ear canal, comfort can be an issue in prolonged use. Besides the pressure exerted against the ear canal wall, the ease of insertion can have an impact on perceived comfort.

Also, hearing protection can take several forms such as earplugs and muffs. Such hearing protection devices attenuate acoustic energy before it reaches the eardrum (tympanum) by creating an insertion loss that is achieved by reflection of the sound waves, dissipation with the device's structure, impedance of the waves through tortuous paths, closing of acoustical valves, and other means. For a hearing protector, the amount of sound pressure level (SPL) reduced, usually measured in decibels (dB), is typically depicted graphically as a function of frequency. Comfort can additionally be an issue for earplugs, as can the eases of insertion.

Many eartips/earplugs are made from soft elastomers and polymers of low durometer for comfort, however soft materials have large coefficient of friction which can cause discomfort during insertion and removal.

### SUMMARY

Devices, system and methods for saturation, coating and preparing eartips is disclosed.

In general eartips are composed of a contact outer portion and a stent portion. The outer portion of an eartip (e.g. outer bulb surface) contacts the ear canal wall when inserted into the ear canal. The inner portion contains a core or channel that can fit on a stent (earphone eartip), while a wider potion aids in insertion onto a stent, or if used as an earplug the core or channel will be at least partially filled in or blocked. Prior to insertion into an ear canal the outer portion and inner portion encapsulate or receive a structure (eartip membrane contacts a structure (e.g., stent part, ledge-movable or part of stent), when inserted or as presented (final form after folded from a negative mold)) a medium (e.g., gas, fluid) that can have an opening aiding molding. Note that the opening can be faced inward toward the ear canal or formed to face toward the ambient environment. Note that the eartip and stent can be fabricated from various materials (e.g., silicon, urethane, rubber) and can include internal channel (tubes). The stent can also be a multi-lumen (i.e., multi-passageway) stent where the channels/tubes are various lumens of the multi-lumen stent, or solid (e.g., earplug stent). Note that the material of the membrane can have different properties from the stent or membrane wall or channel wall. Upon insertion into an ear canal, the ear canal wall pressure on the outer portion of a ridge and the outer portion can move radially and axially to relieve the pressure pressing against the ear canal wall. This is in contrast to foam tips that will always press back radially dependent upon the amount of deformation of the foam. The combination of radial and axial movement of the outer section helps decrease pressure on the ear canal wall and increase contact area also decreasing pressure for a given retaining force.

In one embodiment, an eartip can have an outer portion, an inner portion, and an encapsulated volume formed by the inner and outer portion, wherein the outer portion is designed to contact the ear canal, and wherein the inner portion is designed to fit upon a stent. The eartip can also have a passage to an ambient environment, where the passage is decreased when the eartip is inserted upon a stent or inserted into an ear canal. Also, the encapsulated volume may be at a pressure that is reduced when pressure on the outer surface exceeds a threshold value. Although a specific example of an eartip has been described, embodiments herein should not be interpreted to be limited to any type of eartip.

In another embodiment, an eartip may have an inverted body, where when the inverted body is at least partially folded on itself the inverted body is arranged to a bulbous region sized for insertion in an ear canal, a cavity internal to the bulbous region that holds a gas or enclosed volume, where increasing pressure on the bulbous region releases a portion of the gas. As such and as disclosed herein, gas can escape from enclosed volume or cavity in the eartip to customize the pressure or force provided by the eartip to provide a snug and comfortable tip without the eartip causing user discomfort from excessive pressure or force. Also, the eartip may have a channel wall that forms the cavity with the bulbous region and that forms a core through a stent can be inserted and/or received.

In one embodiment, the eartip can have a plurality of bulbous regions. Likewise, the eartip can have a plurality of cavities, where each cavity of the plurality of cavities is formed by each bulbous region of the plurality of bulbous regions and the channel wall. The eartip can also include a transition region between the plurality of bulbous regions where the transition region has a concavity that is inverse to the convex surface of the bulbous regions. The eartip can also have a lip extending from the channel wall and a sealing section extending from the bulbous region.

In one embodiment, a sealing tip can be located on the sealing section can be provided. In some arrangements, the cavity is sealed by seating the sealing tip against the channel wall. Also, the sealing tip can have a material property between 2 Shore A to 90 Shore A. Also, the eartip can be an earplug with a U.S. Environmental Protection Agency (EPA) Noise Reduction Rating (NRR) rating of at least 3.

Methods of manufacturing an eartip and/or earplug are also disclosed. One method can include printing an eartip as an inverted body, such that when the inverted body is at least partially folded on itself the inverted body is arranged to include one or more of the features disclosed herein. For instance, the eartip can have a bulbous region sized for insertion in an ear canal, a cavity internal to the bulbous region that holds a gas, where increasing pressure on the bulbous region releases a portion of the gas. Further, the method can provide an inverted body eartip with a material property between 2 Shore A to 90 Shore A.

In another embodiment, an earphone system is disclosed. The earphone system can include an earphone and an eartip. The eartip can have structures and functionality described herein and include a reduced skin friction (e.g., less than 2) eartip as a result of methods described by embodiments. The friction of human skin varies, as described in Reference 1 “Tribology of Skin:review and Analysis of Experimental results for the Friction Coefficient of Human Skin” by S. Derler and L. C. Gerhardt, Tribol Lett (2012) 45:1-27, incorporated herein by reference in its entirety. Reference 1 states that friction (e.g., with textiles) actually increases in skin due to water moisture (section 2.4.1, Reference 1), hence a hydrophobic treatment solution (e.g., mineral oil, waxes, olive oil, vegetable oil, teflon coating) should aid in repelling molecular bonding with water, decreasing friction.

In another embodiment, a method of forming an eartip is disclosed. The method can include forming a mold of an unfolded shape of an eartip, providing or supplying a flexible material to the mold for a threshold time and temperature for curing to form a cured inverted eartip, removing the cured inverted eartip and folding at least a portion of the cured inverted eartip to form a final eartip. Forming a mold can be skipped if a mold already has been made. Also, folding at least a portion of cured inverted eartip is optional.

In another embodiment, an earphone is disclosed. The earphone can include a housing, where the housing includes a stent configured to accept or insert into various foam tips, flange tips, and eartips. The earphone can also include an electronics package unit, where the electronics package unit includes an electronic package, and an electronic package housing, wherein the electronics package unit is designed to be independent of the housing. The earphone can also include a key, where the key is part of the housing and is designed to fit with the electronic package unit, where the electronic package includes two microphones and a speaker.

In certain embodiments, the outer portion of an eartip (e.g. the outer surface of an eartip) contacts the ear canal wall when inserted into the ear canal. The inner portion contains a core that can fit on a stent, while a wider portion (inside funnel shape of a ridge) aids in insertion onto a stent. Prior to insertion into an ear canal the outer portion and inner portion encapsulates a medium (e.g., gas, fluid) that can have an opening aiding molding. Upon insertion onto a stent the inner portion can move flexibly outward decreasing the opening, and/or upon inserting into an ear canal, the ear canal wall can press inward on the outer surface toward the stent moving the outer portion of the ridge inward, decreasing the opening. Note that the opening can be faced inward toward the ear canal or formed to face toward the ambient environment. Note that the stent can be fabricated from various materials (e.g., silicon, urethane, rubber) and can include internal channel (tubes). The stent can also be a multi-lumen (i.e., multi-passageway) stent where the channels/tubes are various lumens of the multi-lumen stent. Upon insertion into an ear canal the ear canal wall pressure on the outer portion of a ridge and the outer portion can move radially and axially to relieve the pressure pressing against the ear canal wall. This is in contrast to foam tips that will always press back radially dependent upon the amount of deformation of the foam. The combination of radial and axial movement of the outer section helps decrease pressure on the ear canal wall and increase contact area also decreasing pressure for a given retaining force.

Although the non-limiting examples herein discuss AirTips™ embodiments discussed herein can be applied to eartips and earplugs in general.

These and other features of the eartip, earplug, earphone systems and methods are described in the following detailed description, drawings, and appended claims.

### BRIEF DESCRIPTION OF THE DRAWINGS

FIG. 1 is an angled exploded view of an earphone device illustrating how various parts of the earphone device secure together according to an embodiment of the present disclosure.

FIG. 2 illustrates an earphone device with an eartip according to an embodiment of the present disclosure.

FIG. 3 illustrates a tip with a cross section showing a membrane.

FIGS. 4, 5, 6, 7,8, and 9 illustrate various processes to modify the membrane.

FIG. 10 illustrates a view of a molded eartip prior to folding.

FIG. 11 illustrates the molded eartip of FIG. 10 folded to form a final or insertion arrangement eartip.

FIG. 12 illustrates a view of a molded eartip prior to folding, with the inner membrane surface treated.

FIG. 13 illustrates the molded eartip of FIG. 12 folded to form a final or insertion arrangement eartip, with the treated portion of the membrane facing outward.

FIGS. 14-15 illustrates a view of a molded eartip prior to folding, with the inner membrane surface treated to a selected length.

FIG. 16 illustrates a method of treating an eartip.

FIG. 17 is an image of an eartip being submerged in a treatment solution.

FIG. 18 is an image of an eartip being filled with a treatment solution.

FIG. 19 illustrates a method of treating an eartip.

FIG. 20 illustrates an eartip treated by the method shown in FIG. 19.

FIG. 21 is a schematic diagram of a system for utilizing eartips treated according to an embodiment of the present disclosure.

FIG. 22 is a schematic diagram of a machine in the form of a computer system within which a set of instructions, when executed, may cause the machine to perform any one or more of the methodologies or operations of the systems and methods for utilizing an eartip treated according to embodiments of the present disclosure.

### DETAILED DESCRIPTION OF THE INVENTION

Eartip often are made of soft materials that also have high coefficients of friction (e.g., silicone, high coefficient of friction, greater than 3 (unitless) measured per ASTM standard D1894). A higher coefficient of friction causes the eartip to grab the skin and prevent the ear tip from deforming smoothly and conforming to the ear contour as it is inserted. A surface or eartip material treatment as described below improves the comfort of ear tips and the quality of their fit.

Exemplary embodiments of eartips and earphone devices, and systems and methods therefore are disclosed. The eartips are self-adjusting for the variable sizes of user anatomy. In use, the eartip can adjust radially and/or linearly for maximum comfort with a maintenance force that is less than an insertion force. The structural configuration of the eartip provides such adjustability while providing a flatter high frequency attenuation profile that maintains audio quality in comparison to traditional ear buds. Further, such adjustability is provided for with improved manufacturing techniques

Exemplary embodiments are directed to or can be operatively used on various passive eartips for hearing protection or electronic wired or wireless earpiece devices (e.g., hearing aids, ear monitors, headphones, ear terminal, behind the ear devices or other acoustic devices as known by one of ordinary skill, and equivalents). For example, the earpieces can have one or more transducers (e.g. ambient sound microphone (ASM), ear canal microphone (ECM), ear canal receiver (ECR/SPKR)) for monitoring/providing sound. In all of the examples illustrated and discussed herein, any specific values should be interpreted to be illustrative only and non-limiting. Thus, other examples of the exemplary embodiments could have different values.

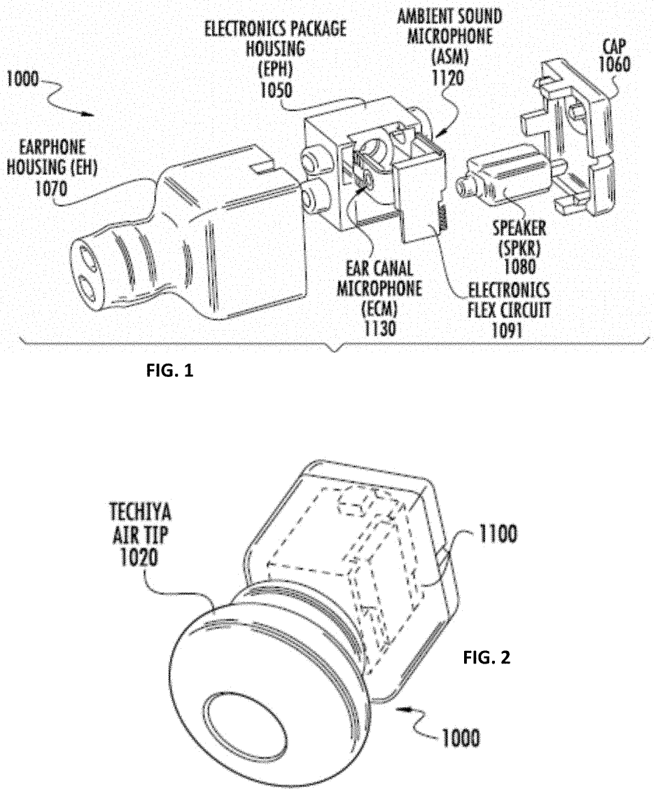

FIG. 1 illustrates various parts that fit within an earphone housing 1070 of a hearbud housing device 1100 that form an earphone device 1000. The parts that fit within the earphone housing 1070 can include an ASM 1120, an ECM 1130, an EPH 1050, a flexible circuit 1091 of an EP 1090, the EP 1090 itself, a SPKR 1080, and a cap 1060.

FIG. 2 illustrates an additional exploded view of a hearbud housing device 1100 with various components labelled and which are configured to be housed within the hearbud housing device 1100. For example, in certain embodiments, the components of the earphone device 1000 can include the hearbud housing device 1100, an earphone housing 1070, a cap 1060, and an electronic package housing 1050, which houses the electronics package (EP) 1090 that can include a speaker (SPKR or ECR) 1080, ambient sound microphone (ASM) 1120, an ear canal microphone (ECM) 1130, and supporting electronics that may form a part of the EP 1090. Note that any microphone that can be used in an earphone can be used for the ASM 1120 and ECM 1130. Additionally, any speaker that can be used in earphones can be used for the SPKR 1080 in the earphone device 1000.

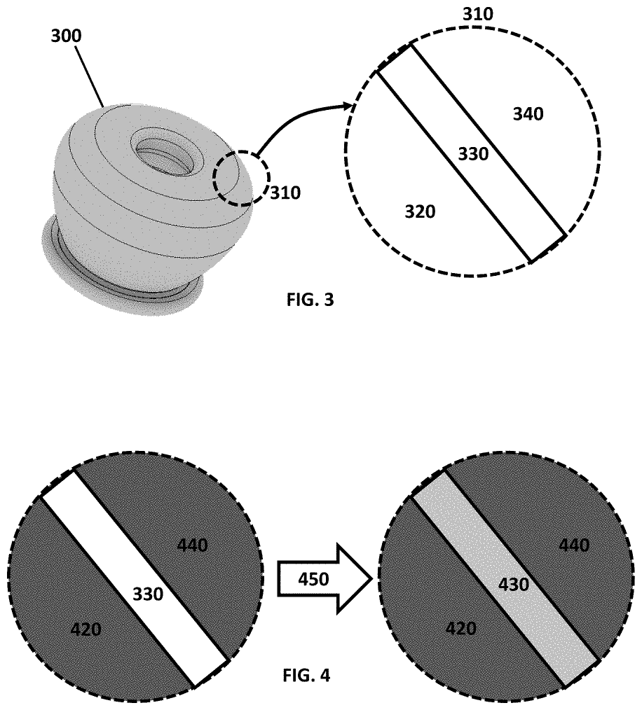

FIG. 3 illustrates an eartip 300 with a cross sectional segment 310 showing a portion of the membrane 330 separating the inside 320 from the outside 340. The discussions that follow with regards to FIGS. 4 to 9 describe various levels of treatment solution (e.g., lubricant penetration, antibacterial solution) or coatings that can occur. For example FIG. 4 illustrates membrane 330 before treatment 450 surrounded by treatment solution 420, 440, where the treatment 450 results in a modified membrane 430 with embedded/absorbed solution. Methods of treatment are discussed in more detail with regards to FIGS. 12 and 16 below. With regards to FIG. 4, the molded eartip 300, can be dipped into treatment solution encompassing both sides (420, 440) for a period of time (e.g., 3-120 mins) at a particular temperature (e.g., room temp+1 C to 200 C). Placing the molded eartip 300 into a heated treatment solution, expands the eartip material, making it more susceptible to intake (absorption) of whatever solution the eartip 300 is in. Upon removal, when the eartip 300 reaches room temperature, the treatment solution will seep gradually from the eartip 300 due to the difference in absorption saturation at different temperatures of the eartip material. If the treatment solution is a lubricant, then the eartip 300 will be self lubricating for a period of time (e.g., 3 days to 6 months). Note that the treatment solution can also be an antibacterial, that helps keep the eartip 300 clean. The treatment solution (e.g., treatment of the eartip 300 material not a patient) can vary depending upon need, for example a lubricant (e.g., mineral oil), an antibacterial, an anti-viral, even medicines or vitamins that can be absorbed through the skin of the ear canal to treat a patient. Note that if a device having the eartip 300 on it has a biometric sensor(s) that needs better connection to the skin, the eartips 300 can secrete a substance that improve biometric pickup between the eartip 300 and the skin (e.g., one that improves the transmission of pickup signals such as infrared or acoustic energy). Another method of embedding treatment solution in the eartip material is to mix a very small portion with the resin before curing. For example, 0.02% to 25% by weight. For example, a 4-5% by weight of mineral oil mixed with High Strength 2 (Alumilite™) results in a cured eartip that reduces both kinetic and static friction coefficients. Note that the mixing can occur at elevated temperatures (e.g., room temp+1 C to 200 C) or even at room temperature, then allowed to cure. Note that various methods of curing, e.g., two or three part resin mixing, UV curing and other methods can also be used. Treated membrane 430 has been penetrated throughout to various levels based upon depth from the membranes 430 surface.

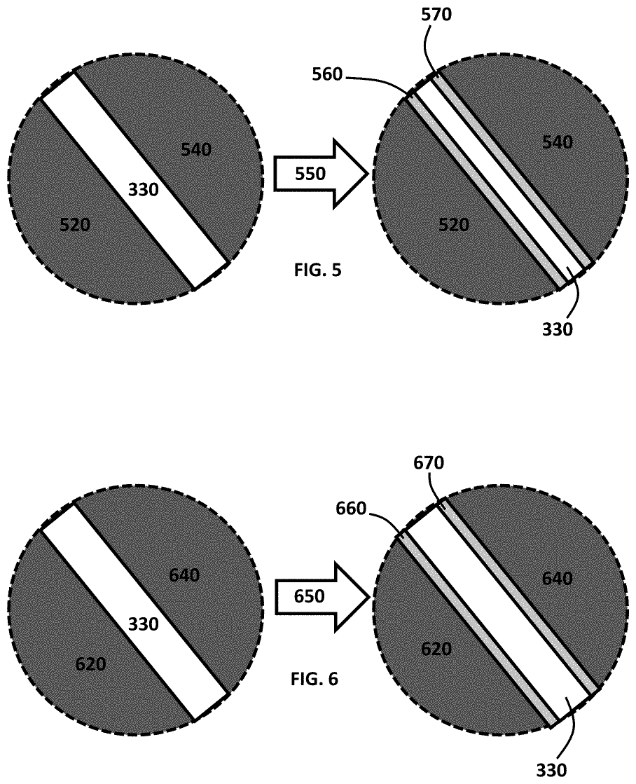

FIG. 5 illustrates membrane 330 before treatment 550 surrounded by treatment solution 520, 540, where the treatment 550 results in a modified membrane 530 with embedded/absorbed solution. The solution penetrates membrane 330 to a depth to form penetrated layers 560 and 570. The thickness of layers 560 and 570 depend on the concentrations of solutions 520, 540, on either side of membrane 330, as well as temperatures of 520 and 540. For example if the solution concentration (e.g., % (by weight or volume) mineral oil in solution) is greater in solution 520 than 540 (o vice versa), then over a period of time (e.g., 1 min to 24 hours) the thickness of penetration layer 560 will be greater than penetration layer 570.

FIG. 6 illustrates membrane 330 before treatment 650 surrounded by treatment solution 620, 640, where the treatment 650 results in a coating(s) 620, 640 on membrane 330. The solution coats membrane 330 a thickness to form coating layers 660 and 670. The thickness of layers 660 and 670 depend on the concentrations of solutions 620, 640, on either side of membrane 330, as well as temperatures of 620 and 640. Note that in addition to the coating layers 660 and 670, the solution may also penetrate a depth into membrane 330. Another method of coating the eartips 300 is to use a semi-conductive eartip 300 material and dip the eartip 300 into a solution with an applied voltage on the eartip 300 to combine with the electrode (e.g., in this case the eartip membrane 330). For example, suppose we wish to coat a layer of antibacterial copper on the outer surface of the eartip 300. Then solution 640 could be a 1.0 M (molar concentration) CuSO4 in 1.0 M H2SO4, where the eartip 300 (made of semi conductive to conductive material, e.g., copper nanoparticles mixed with precured resin) acts as the cathode. Application of a voltage between the eartip 300 and an anode (e.g., of copper) coats a thin layer of copper (e.g., 670 for solution 640, if both solutions 620, 640 contain electrolyte then 660 and 670). Note that if the eartip 300 is conductive it can connect with an antenna in an earphone to improve reception so that the head of a user acts as a counterpoise.

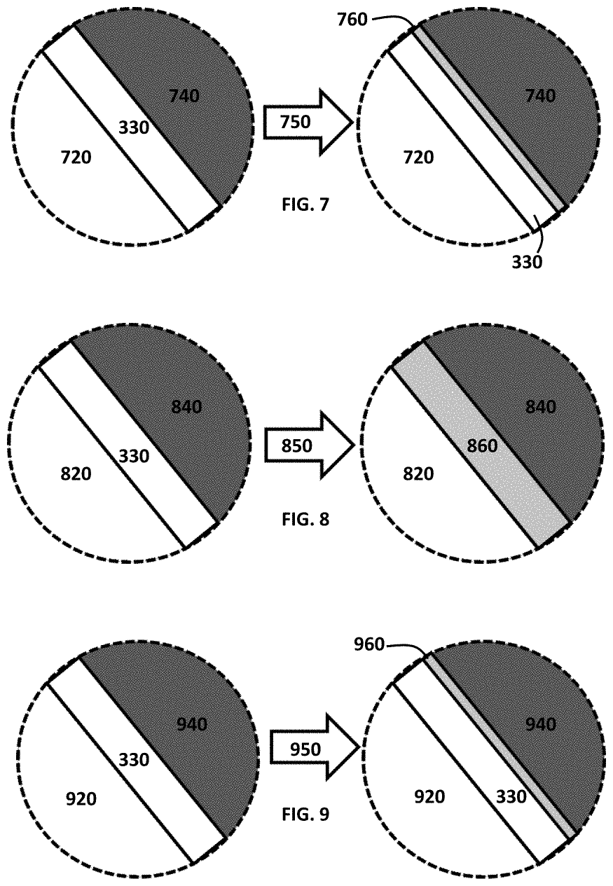

FIGS. 7-9 illustrate various types of coatings, saturations, with the treatment solution on one side (e.g., 740, 840, 940) of membrane 330. In FIG. 7 treatment 750 results in penetration of the treatment solution 740 into a depth of membrane 330 by a thickness 760, dependent on temperature of 330 and/or 740, pressure of 740 (e.g., 1 to 5 atmospheres), and treatment solution concentration. Treatment 850 results in the treatment solution on side 840 and not side 820, being roughly homogeneous throughout the membrane 860, also referred to as full penetration of membrane 330 with treatment solution. Note that another way to get a homogenous distribution (not necessarily fully saturated) of treatment solution within the membrane is to combine the treatment solution with the epoxy/resin before curing by weight or volume % as discussed above. Treatment 950 results in a layer 960 deposited upon membrane 330 where the treatment solution is on side 940 compared to side 920.

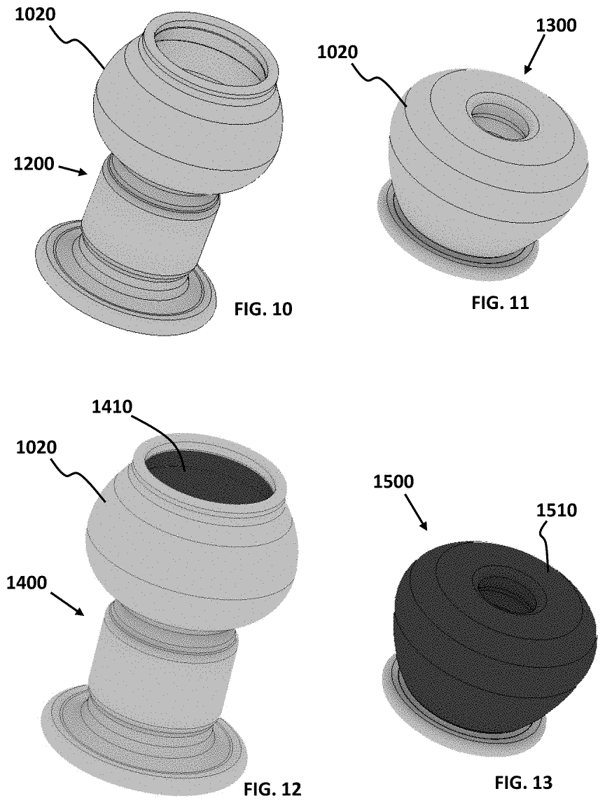

FIG. 10 illustrates 1200 a view of a molded eartip 1020 prior to folding. Molding the eartip unfolded enables molding, then folding the tip region results in a nearly closed cavity which is very difficult to mold outright. FIG. 11 illustrates 1300 the molded eartip of FIG. 10 folded to form a final or insertion arrangement eartip 1020. The material of the eartip 1020 is often stable to high temperatures (e.g., greater than 100 C), therefore to treat the outside surface (outside after folding) the treatment solution can be pooled into the cavity 1410 prior to folding, then heated (e.g., from about 40 C to 200 C) for a predetermined time (e.g., 1 min to 2 hours). FIG. 12 illustrates 1400 a view of a molded eartip 1020 prior to folding, with the inner membrane surface treated 1410. The treated eartip is then cleaned and folded 1500 (FIG. 12) so that the outside surface with the coating or penetrated surface 1510. FIG. 13 illustrates 1500 the molded eartip 1020 of FIG. 12 folded to form a final or insertion arrangement eartip, with the treated portion 1510 of the membrane facing outward.

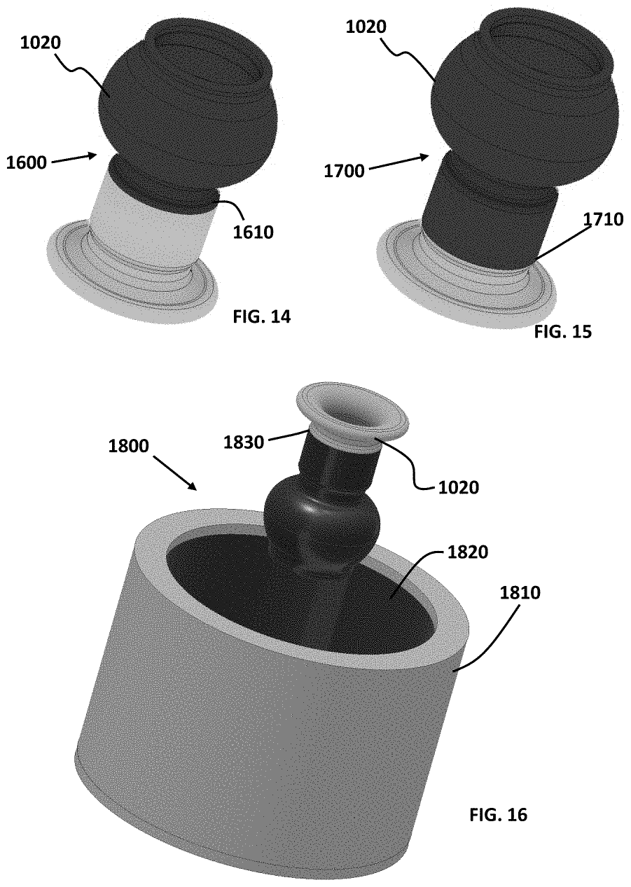

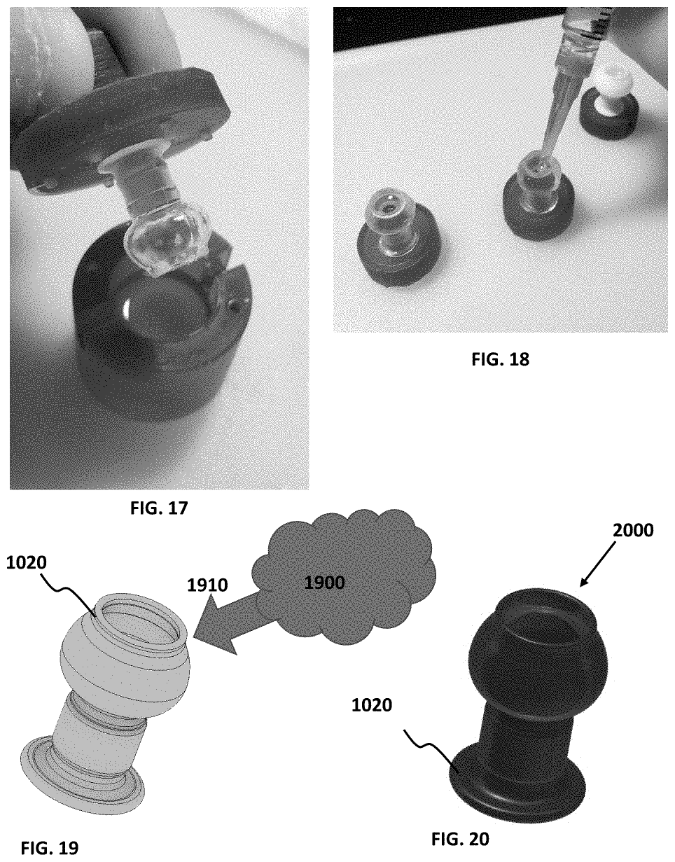

FIGS. 14-15 illustrates (1600,1700) a view of a molded eartip 1020 prior to folding, with the inner membrane surface (becomes the inner surface when folded) treated to a selected length (1610, 1710, 1830), for example by dipping the untreated eartip 1020 into a heated treatment solution 1820 (FIG. 16). FIG. 16 illustrates 1800 a method of treating an eartip 1020 by dipping the eartip 1020 to a depth to a line 1830 into treatment solution 1820 in container 1810. FIG. 17 is an image of an eartip being submerged in a treatment solution. FIG. 18 is an image of an eartip being filled with a treatment solution.

FIG. 19 illustrates a method of treating an eartip 1020, where vapor coating 1900 is coated via vapor deposition 1910 onto the eartip 1020 forming a coated eartip 2000. FIG. 20 illustrates 2000 an eartip 1020 treated by the method shown in FIG. 19. Note that the eartip can be a polymer or elastomer (e.g. silicone, polyurethane, and thermoplastic) that is cured as discussed above and as known by one of ordinary skill in eartip design and manufacturing. Also note that any type of treatment solution (e.g., mineral oil, antibacterial solution) can be used.

An alternative method to reduce the friction of material used for the eartip is to use a very smooth mold surface so that the silicone or soft material touching the surface of the mold has very small variations across its surface., for example cover the mold surfaces with a Teflon spray that bonds to the mold surface prior to molding. Then when the mold material is inserted into the mold, the Teflon coated side will smooth out the mold material decreasing the skin friction of that side of the eartip.

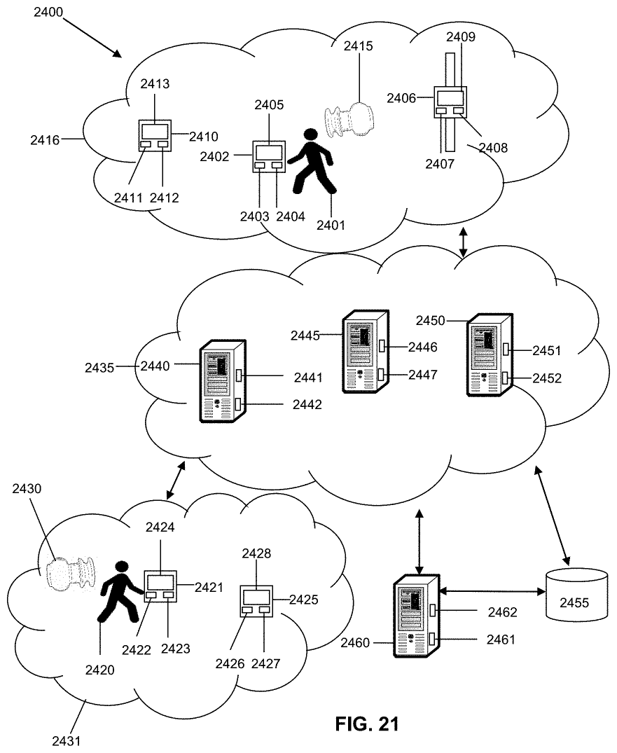

As shown in FIG. 21, a system 2400 and methods for utilizing eartips and/or earphone devices are disclosed.

The system 2400 may be configured to support, but is not limited to supporting, data and content services, audio processing applications and services, audio output and/or input applications and services, applications and services for transmitting and receiving audio content, authentication applications and services, computing applications and services, cloud computing services, internet services, satellite services, telephone services, software as a service (SaaS) applications, platform-as-a-service (PaaS) applications, gaming applications and services, social media applications and services, productivity applications and services, voice-over-internet protocol (VoIP) applications and services, speech-to-text translation applications and services, interactive voice applications and services, mobile applications and services, and any other computing applications and services. The system may include a first user 2401, who may utilize a first user device 2402 to access data, content, and applications, or to perform a variety of other tasks and functions. As an example, the first user 2401 may utilize first user device 2402 to access an application (e.g. a browser or a mobile application) executing on the first user device 2402 that may be utilized to access web pages, data, and content associated with the system 2400. In certain embodiments, the first user 2401 may be any type of user that may potentially desire to listen to audio content, such as from, but not limited to, a music playlist accessible via the first user device 2402, a telephone call that the first user 2401 is participating in, audio content occurring in an environment in proximity to the first user 2401, any other type of audio content, or a combination thereof. For example, the first user 2401 may be an individual that may be participating in a telephone call with another user, such as second user 2420.

The first user device 2402 utilized by the first user 2401 may include a memory 2403 that includes instructions, and a processor 2404 that executes the instructions from the memory 2403 to perform the various operations that are performed by the first user device 2402. In certain embodiments, the processor 2404 may be hardware, software, or a combination thereof. The first user device 2402 may also include an interface 2405 (e.g. screen, monitor, graphical user interface, etc.) that may enable the first user 2401 to interact with various applications executing on the first user device 2402, to interact with various applications executing within the system 2400, and to interact with the system 2400 itself In certain embodiments, the first user device 2402 may include any number of transducers, such as, but not limited to, microphones, speakers, any type of audio-based transducer, any type of transducer, or a combination thereof. In certain embodiments, the first user device 2402 may be a computer, a laptop, a tablet device, a phablet, a server, a mobile device, a smartphone, a smart watch, and/or any other type of computing device. Illustratively, the first user device 2402 is shown as a mobile device in FIG. 24. The first user device 2402 may also include a global positioning system (GPS), which may include a GPS receiver and any other necessary components for enabling GPS functionality, accelerometers, gyroscopes, sensors, and any other componentry suitable for a mobile device.

In addition to using first user device 2402, the first user 2401 may also utilize and/or have access to a second user device 2406 and a third user device 2410. As with first user device 2402, the first user 2401 may utilize the second and third user devices 2406, 2410 to transmit signals to access various online services and content. The second user device 2406 may include a memory 2407 that includes instructions, and a processor 2408 that executes the instructions from the memory 2407 to perform the various operations that are performed by the second user device 2406. In certain embodiments, the processor 2408 may be hardware, software, or a combination thereof. The second user device 2406 may also include an interface 2409 that may enable the first user 2401 to interact with various applications executing on the second user device 2406 and to interact with the system 2400. In certain embodiments, the second user device 2406 may include any number of transducers, such as, but not limited to, microphones, speakers, any type of audio-based transducer, any type of transducer, or a combination thereof. In certain embodiments, the second user device 2406 may be and/or may include a computer, any type of sensor, a laptop, a set-top-box, a tablet device, a phablet, a server, a mobile device, a smartphone, a smart watch, and/or any other type of computing device. Illustratively, the second user device 2402 is shown as a smart watch device in FIG. 24.

The third user device 2410 may include a memory 2411 that includes instructions, and a processor 2412 that executes the instructions from the memory 2411 to perform the various operations that are performed by the third user device 2410. In certain embodiments, the processor 2412 may be hardware, software, or a combination thereof. The third user device 2410 may also include an interface 2413 that may enable the first user 2401 to interact with various applications executing on the second user device 2406 and to interact with the system 2400. In certain embodiments, the third user device 2410 may include any number of transducers, such as, but not limited to, microphones, speakers, any type of audio-based transducer, any type of transducer, or a combination thereof. In certain embodiments, the third user device 2410 may be and/or may include a computer, any type of sensor, a laptop, a set-top-box, a tablet device, a phablet, a server, a mobile device, a smartphone, a smart watch, and/or any other type of computing device. Illustratively, the third user device 2410 is shown as a smart watch device in FIG. 24.

The first, second, and/or third user devices 2402, 2406, 2410 may belong to and/or form a communications network 2416. In certain embodiments, the communications network 2416 may be a local, mesh, or other network that facilitates communications among the first, second, and/or third user devices 2402, 2406, 2410 and/or any other devices, programs, and/or networks of system 2400 or outside system 2400. In certain embodiments, the communications network 2416 may be formed between the first, second, and third user devices 2402, 2406, 2410 through the use of any type of wireless or other protocol and/or technology. For example, the first, second, and third user devices 2402, 2406, 2410 may communicate with one another in the communications network 2416, such as by utilizing Bluetooth Low Energy (BLE), classic Bluetooth, ZigBee, cellular, NFC, Wi-Fi, Z-Wave, ANT+, IEEE 802.15.4, IEEE 802.22, ISA100a, infrared, ISM band, RFID, UWB, Wireless HD, Wireless USB, any other protocol and/or wireless technology, satellite, fiber, or any combination thereof. Notably, the communications network 2416 may be configured to communicatively link with and/or communicate with any other network of the system 2400 and/or outside the system 2400.

The system 2400 may also include an earphone device 2415, which the first user 2401 may utilize to hear and/or audition audio content, transmit audio content, receive audio content, experience any type of content, process audio content, adjust audio content, store audio content, perform any type of operation with respect to audio content, or a combination thereof. The earphone device 2415 may be an earpiece, a hearing aid, an ear monitor, an ear terminal, a behind-the-ear device, any type of acoustic device, or a combination thereof. The earphone device 2415 may include any type of component utilized for any type of earpiece. In certain embodiments, the earphone device 2415 may include any number of ambient sound microphones that may be configured to capture and/or measure ambient sounds and/or audio content occurring in an environment that the earphone device 2415 is present in and/or is proximate to. In certain embodiments, the ambient sound microphones may be placed at a location or locations on the earphone device 2415 that are conducive to capturing and measuring ambient sounds occurring in the environment. For example, the ambient sound microphones may be positioned in proximity to a distal end (e.g. the end of the earphone device 2415 that is not inserted into the first user's 2401 ear) of the earphone device 2415 such that the ambient sound microphones are in an optimal position to capture ambient or other sounds occurring in the environment. In certain embodiments, the earphone device 2415 may include any number of ear canal microphones, which may be configured to capture and/or measure sounds occurring in an ear canal of the first user 2401 or other user wearing the earphone device 2415. In certain embodiments, the ear canal microphones may be positioned in proximity to a proximal end (e.g. the end of the earphone device 2415 that is inserted into the first user's 2401 ear) of the earphone device 2415 such that sounds occurring in the ear canal of the first user 2401 may be captured more readily.

The earphone device 2415 may also include any number of transceivers, which may be configured transmit signals to and/or receive signals from any of the devices in the system 2400. In certain embodiments, a transceiver of the earphone device 2415 may facilitate wireless connections and/or transmissions between the earphone device 2415 and any device in the system 2400, such as, but not limited to, the first user device 2402, the second user device 2406, the third user device 2410, the fourth user device 2421, the fifth user device 2425, the earphone device 2430, the servers 2440, 2445, 2450, 2460, and the database 2455. The earphone device 2415 may also include any number of memories for storing content and/or instructions, processors that execute the instructions from the memories to perform the operations for the earphone device 2415, and/or any type integrated circuit for facilitating the operation of the earphone device 2415. In certain embodiments, the processors may comprise, hardware, software, or a combination of hardware and software. The earphone device 2415 may also include one or more ear canal receivers, which may be speakers for outputting sound into the ear canal of the first user 2401. The ear canal receivers may output sounds obtained via the ear canal microphones, ambient sound microphones, any of the devices in the system 2400, from a storage device of the earphone device 2415, or any combination thereof.

The ear canal receivers, ear canal microphones, transceivers, memories, processors, integrated circuits, and/or ear canal receivers may be affixed to an electronics package that includes a flexible electronics board. The earphone device 2415 may include an electronics packaging housing that may house the ambient sound microphones, ear canal microphones, ear canal receivers (i.e. speakers), electronics supporting the functionality of the microphones and/or receivers, transceivers for receiving and/or transmitting signals, power sources (e.g. batteries and the like), any circuitry facilitating the operation of the earphone device 2415, or any combination thereof. The electronics package including the flexible electronics board may be housed within the electronics packaging housing to form an electronics packaging unit. The earphone device 2415 may further include an earphone housing, which may include receptacles, openings, and/or keyed recesses for connecting the earphone housing to the electronics packaging housing and/or the electronics package. For example, nozzles of the electronics packaging housing may be inserted into one or more keyed recesses of the earphone housing so as to connect and secure the earphone housing to the electronics packaging housing. When the earphone housing is connected to the electronics packaging housing, the combination of the earphone housing and the electronics packaging housing may form the earphone device 2415. The earphone device 2415 may further include a cap for securing the electronics packaging housing, the earphone housing, and the electronics package together to form the earphone device 2415.

In certain embodiments, the earphone device 2415 may be configured to have any number of changeable tips, which may be utilized to facilitate the insertion of the earphone device 2415 into an ear aperture of an ear of the first user 2401, secure the earphone device 2415 within the ear canal of an ear of the first user 2401, and/or to isolate sound within the ear canal of the first user 2401. The tips may be foam tips, which may be affixed onto an end of the earphone housing of the earphone device 2415, such as onto a stent and/or attachment mechanism of the earphone housing. In certain embodiments, the tips may be any type of eartip as disclosed and described in the present disclosure. The eartips as disclosed in the present disclosure may be configured to facilitate distributed reduced contact force, sound isolation for sound in the ear canal of the first user 2401 (i.e. between the ambient environment and the ear canal environment within an ear of the first user 2401), mold into a variety of forms and/or positions, encapsulate volumes upon insertion into an ear aperture of the first user 2401, have a pressure adjusting design, facilitate notched stent retention (i.e. on a stent of the earphone housing), facilitate stent insertion into an ear canal of the first user 2401 via an ear aperture of the first user 2401, or any combination thereof In certain embodiments, the eartip may be designed to provide sound isolation capability that is at least as effective as conventional foam and/or flange tips. Notably, the eartips may be manufactured and configured to be made in any desired size specifications and/or materials, and may be tailored to each individual user, such as first user 2401. In contrast to conventional foam or flange tips, an eartip according to the present disclosure may be adjusted for size without having to substitute the eartip with another eartip, may have an EPA NRR rating of NRR=18, may have a unique flatter high frequency attenuation profile so as to maintain audio quality, may have ease of manufacturability, and may be designed to distribute contact force and minimize radial force against a user's ear canal walls when positioned in a user's ear canal. Additionally, an eartip according to the present disclosure may be made of a non-porous material that is not closed cell foam or open cell foam.

In certain embodiments, the eartip may be designed so that the earphone device's 2415 retention force on the ear canal walls of the first user 2401 may be distributed over a larger area than traditional foam or flange tips allow, thereby reducing the pressure on the ear canal walls of the first user 2401. Unlike foam tips, which primarily provide a restoring radial force that exerts pressure against the ear canal walls of a user, the eartip is designed to move both radially and axially, which allows for more give and redistribution of contact over a larger area, and, thus, decreases the retention pressure. As a result, this allows for increased comfort for the user and allows the user to utilize the eartip for an extended period of time when compared to traditional foam and/or flange tips. In certain embodiments, the eartip utilized with the earphone device 2415 may be configured to encapsulate a volume of gas and/or liquid. In either case (i.e. gas or liquid), the bulk of sound isolation provided by the eartip is achieved through the reflection of ambient sound waves so that the encapsulated volume can be low mass. In certain embodiments, portions of the eartip may encapsulate a volume with the ability to release volume when pressed upon without having to incorporate complicated valves. The encapsulated volume may be achieved by the ear canal wall pressing radially and/or axially against the outer surfaces of the eartip, which may force the outer portion of the eartip to seal with the inner portion of the eartip. In certain embodiments, the inner portion of the eartip may be small than the outer diameter of the stent of the earphone housing upon which the eartip is placed so that upon insertion of the eartip on the stent, the inner portion stretches outward to meet the outer surface of the eartip, which further facilitates the sealing of the ear canal of the first user 2401.

In certain embodiments, the stent of the eartip, over which the eartip is placed, may be designed to have a smaller diameter front end and a larger diameter middle section to promote retention of the eartip on the stent itself. In certain embodiments, a portion of the eartip may have an inner core diameter that is smaller than the stent outer diameter so that the eartip provides radial compression upon the stent so as to enhance sealing and to add friction to prevent axial slippage within the ear canal of the first user 2401. In certain embodiments, an increased mid-section inner core diameter of the eartip may be utilized (i.e. larger than the smaller inner core diameter of the eartip), which may be configured to line up with the mid-section outer diameter of the stent of the earphone housing of the earphone device 2415. This may provide axial stability for the earphone device 2415, while simultaneously preventing axial slippage from the ear canal of the first user 2401. In certain embodiments, the eartip may have an insertion end that has a funnel shape, which aids in inserting the eartip onto the stent of the earphone housing of the earphone device 2415.

In certain embodiments, the eartip has a configuration that applies minimal force against the first user's 2401 ear canal. Additionally, the eartip can seal the first user's 2401 ear canal by providing at least 15 dB of attenuation across frequency. To facilitate manufacturability, the eartip may be molded inverted, thereby allowing inexpensive mass production. Lips of the eartip may then be folded to contact ledges to for the eartip that may be utilized by the first user 2401. Sealing and comfort depend upon an accurate fit within the first user's 2401 ear canal, and, as a result, eartips according to the present disclosure may be manufactured in several single sizes, and, because of the unique design of the eartips, a single eartip may be adjusted to fit multiple sizes, which minimizes manufacturing costs, while allowing for more flexibility, versatility, and for a greater number of sizes for the eartip. Notably, any of the features of any of the eartips described in the present disclosure may be combined and/or interchanged with any other eartips described in the present disclosure. Furthermore, the shape, size, features and/or functionality of any of the components of the earphone device and/or hearbud housing device described in the present disclosure may be modified for each particular user for the shape and size of each user's ear aperture and/or ear canal, or a combination thereof.

Notably, in experiments conducted using the eartip, the experiments have shown that the eartip allows for similar levels of sound isolation when compared to conventional foam and/or flange tips. For example, experiments have shown that the eartips provided in the present disclosure provided an NRR of 18 with a generally flat high frequency profile. A flat attenuation profile maintains an ambient environment's frequency profile when level reduced by the attenuation, which can be useful in maintaining the quality of ambient speech and music (or other audio content) during the level reduction process.

In further embodiments, the eartip may be configured to have an open configuration prior to insertion onto a stent of the earphone housing and/or the earphone device 2415 itself. By having an open configuration, the eartip may be mass produced using conventional molding techniques and/or by utilizing 3D commercial printers. The open configuration of the eartip also facilitates molding, and can be 3D printed, where the open configuration allows for resin removal. For example, resin removal may be achieved by utilizing commercial 3D printers that allow the use of lower durometer materials, such as Stratasys machines and the like. In certain embodiments, since the eartip has an open configuration, which is then sealed, any additional pressure can force encapsulated gas out of the eartip relieving the feedback pressure so as to keep the comfort level for the first user 2401 relatively stable.

In addition to the first user 2401, the system 2400 may include a second user 2420, who may utilize a fourth user device 2421 to access data, content, and applications, or to perform a variety of other tasks and functions. Much like the first user 2401, the second user 2420 may be may be any type of user that may potentially desire to listen to audio content, such as from, but not limited to, a storage device of the fourth user device 2421, a telephone call that the second user 2420 is participating in, audio content occurring in an environment in proximity to the second user 2420, any other type of audio content, or a combination thereof. For example, the second user 2420 may be an individual that may be listening to songs stored in a playlist that resides on the fourth user device 2421. Also, much like the first user 2401, the second user 2420 may utilize fourth user device 2421 to access an application (e.g. a browser or a mobile application) executing on the fourth user device 2421 that may be utilized to access web pages, data, and content associated with the system 2400. The fourth user device 2421 may include a memory 2422 that includes instructions, and a processor 2423 that executes the instructions from the memory 2422 to perform the various operations that are performed by the fourth user device 2421. In certain embodiments, the processor 2423 may be hardware, software, or a combination thereof. The fourth user device 2421 may also include an interface 2424 (e.g. a screen, a monitor, a graphical user interface, etc.) that may enable the second user 2420 to interact with various applications executing on the fourth user device 2421, to interact with various applications executing in the system 2400, and to interact with the system 2400. In certain embodiments, the fourth user device 2421 may include any number of transducers, such as, but not limited to, microphones, speakers, any type of audio-based transducer, any type of transducer, or a combination thereof. In certain embodiments, the fourth user device 2421 may be a computer, a laptop, a tablet device, a phablet, a server, a mobile device, a smartphone, a smart watch, and/or any other type of computing device. Illustratively, the fourth user device 2421 may be a computing device in FIG. 24. The fourth user device 2421 may also include any of the componentry described for first user device 2402, the second user device 2406, and/or the third user device 2410. In certain embodiments, the fourth user device 2421 may also include a global positioning system (GPS), which may include a GPS receiver and any other necessary components for enabling GPS functionality, accelerometers, gyroscopes, sensors, and any other componentry suitable for a computing device.

In addition to using fourth user device 2421, the second user 2420 may also utilize and/or have access to a fifth user device 2425. As with fourth user device 2421, the second user 2420 may utilize the fourth and fifth user devices 2421, 2425 to transmit signals to access various online services and content. The fifth user device 2425 may include a memory 2426 that includes instructions, and a processor 2427 that executes the instructions from the memory 2426 to perform the various operations that are performed by the fifth user device 2425. In certain embodiments, the processor 2427 may be hardware, software, or a combination thereof. The fifth user device 2425 may also include an interface 2428 that may enable the second user 2420 to interact with various applications executing on the fifth user device 2425 and to interact with the system 2400. In certain embodiments, the fifth user device 2425 may include any number of transducers, such as, but not limited to, microphones, speakers, any type of audio-based transducer, any type of transducer, or a combination thereof. In certain embodiments, the fifth user device 2425 may be and/or may include a computer, any type of sensor, a laptop, a set-top-box, a tablet device, a phablet, a server, a mobile device, a smartphone, a smart watch, and/or any other type of computing device. Illustratively, the fifth user device 2425 is shown as a tablet device in FIG. 21.

The fourth and fifth user devices 2421, 2425 may belong to and/or form a communications network 2431. In certain embodiments, the communications network 2431 may be a local, mesh, or other network that facilitates communications between the fourth and fifth user devices 2421, 2425, and/or any other devices, programs, and/or networks of system 2400 or outside system 2400. In certain embodiments, the communications network 2431 may be formed between the fourth and fifth user devices 2421, 2425 through the use of any type of wireless or other protocol and/or technology. For example, the fourth and fifth user devices 2421, 2425 may communicate with one another in the communications network 2416, such as by utilizing BLE, classic Bluetooth, ZigBee, cellular, NFC, Wi-Fi, Z-Wave, ANT+, IEEE 802.15.4, IEEE 802.22, ISA100a, infrared, ISM band, RFID, UWB, Wireless HD, Wireless USB, any other protocol and/or wireless technology, satellite, fiber, or any combination thereof. Notably, the communications network 2431 may be configured to communicatively link with and/or communicate with any other network of the system 2400 and/or outside the system 2400.

Much like first user 2401, the second user 2420 may have his or her own earphone device 2430. The earphone device 2430 may be utilized by the second user 2420 to hear and/or audition audio content, transmit audio content, receive audio content, experience any type of content, process audio content, adjust audio content, store audio content, perform any type of operation with respect to audio content, or a combination thereof. The earphone device 2430 may be an earpiece, a hearing aid, an ear monitor, an ear terminal, a behind-the-ear device, any type of acoustic device, or a combination thereof. The earphone device 2430 may include any type of component utilized for any type of earpiece, and may include any of the features, functionality and/or components described and/or usable with earphone device 2415. For example, earphone device 2430 may include any number of transceivers, ear canal microphones, ambient sound microphones, processors, memories, housings, eartips, foam tips, flanges, any other component, or any combination thereof.

In certain embodiments, the first, second, third, fourth, and/or fifth user devices 2402, 2406, 2410, 2421, 2425 and/or earphone devices 2415, 2430 may have any number of software applications and/or application services stored and/or accessible thereon. For example, the first and second user devices 2402, 2411 may include applications for processing audio content, applications for playing, editing, transmitting, and/or receiving audio content, streaming media applications, speech-to-text translation applications, cloud-based applications, search engine applications, natural language processing applications, database applications, algorithmic applications, phone-based applications, product-ordering applications, business applications, e-commerce applications, media streaming applications, content-based applications, database applications, gaming applications, internet-based applications, browser applications, mobile applications, service-based applications, productivity applications, video applications, music applications, social media applications, presentation applications, any other type of applications, any types of application services, or a combination thereof In certain embodiments, the software applications and services may include one or more graphical user interfaces so as to enable the first and second users 2401, 2420 to readily interact with the software applications. The software applications and services may also be utilized by the first and second users 2401, 2420 to interact with any device in the system 2400, any network in the system 2400 (e.g. communications networks 2416, 2431, 2435), or any combination thereof. For example, the software applications executing on the first, second, third, fourth, and/or fifth user devices 2402, 2406, 2410, 2421, 2425 and/or earphone devices 2415, 2430 may be applications for receiving data, applications for storing data, applications for auditioning, editing, storing and/or processing audio content, applications for receiving demographic and preference information, applications for transforming data, applications for executing mathematical algorithms, applications for generating and transmitting electronic messages, applications for generating and transmitting various types of content, any other type of applications, or a combination thereof. In certain embodiments, the first, second, third, fourth, and/or fifth user devices 2402, 2406, 2410, 2421, 2425 and/or earphone devices 2415, 2430 may include associated telephone numbers, internet protocol addresses, device identities, or any other identifiers to uniquely identify the first, second, third, fourth, and/or fifth user devices 2402, 2406, 2410, 2421, 2425 and/or earphone devices 2415, 2430 and/or the first and second users 2401, 2420. In certain embodiments, location information corresponding to the first, second, third, fourth, and/or fifth user devices 2402, 2406, 2410, 2421, 2425 and/or earphone devices 2415, 2430 may be obtained based on the internet protocol addresses, by receiving a signal from the first, second, third, fourth, and/or fifth user devices 2402, 2406, 2410, 2421, 2425 and/or earphone devices 2415, 2430 or based on profile information corresponding to the first, second, third, fourth, and/or fifth user devices 2402, 2406, 2410, 2421, 2425 and/or earphone devices 2415, 2430.

The system 2400 may also include a communications network 2435. The communications network 2435 may be under the control of a service provider, the first and/or second users 2401, 2420, any other designated user, or a combination thereof. The communications network 2435 of the system 2400 may be configured to link each of the devices in the system 2400 to one another. For example, the communications network 2435 may be utilized by the first user device 2402 to connect with other devices within or outside communications network 2435. Additionally, the communications network 2435 may be configured to transmit, generate, and receive any information and data traversing the system 2400. In certain embodiments, the communications network 2435 may include any number of servers, databases, or other componentry. The communications network 2435 may also include and be connected to a mesh network, a local network, a cloud-computing network, an IMS network, a VoIP network, a security network, a VoLTE network, a wireless network, an Ethernet network, a satellite network, a broadband network, a cellular network, a private network, a cable network, the Internet, an internet protocol network, MPLS network, a content distribution network, any network, or any combination thereof. Illustratively, servers 2440, 2445, and 2450 are shown as being included within communications network 2435. In certain embodiments, the communications network 2435 may be part of a single autonomous system that is located in a particular geographic region, or be part of multiple autonomous systems that span several geographic regions.

Notably, the functionality of the system 2400 may be supported and executed by using any combination of the servers 2440, 2445, 2450, and 2460. The servers 2440, 2445, and 2450 may reside in communications network 2435, however, in certain embodiments, the servers 2440, 2445, 2450 may reside outside communications network 2435. The servers 2440, 2445, and 2450 may provide and serve as a server service that performs the various operations and functions provided by the system 2400. In certain embodiments, the server 2440 may include a memory 2441 that includes instructions, and a processor 2442 that executes the instructions from the memory 2441 to perform various operations that are performed by the server 2440. The processor 2442 may be hardware, software, or a combination thereof. Similarly, the server 2445 may include a memory 2446 that includes instructions, and a processor 2447 that executes the instructions from the memory 2446 to perform the various operations that are performed by the server 2445. Furthermore, the server 2450 may include a memory 2451 that includes instructions, and a processor 2452 that executes the instructions from the memory 2451 to perform the various operations that are performed by the server 2450. In certain embodiments, the servers 2440, 2445, 2450, and 2460 may be network servers, routers, gateways, switches, media distribution hubs, signal transfer points, service control points, service switching points, firewalls, routers, edge devices, nodes, computers, mobile devices, or any other suitable computing device, or any combination thereof In certain embodiments, the servers 2440, 2445, 2450 may be communicatively linked to the communications network 2435, the communications network 2416, the communications network 2431, any network, any device in the system 2400, any program in the system 2400, or any combination thereof.

The database 2455 of the system 2400 may be utilized to store and relay information that traverses the system 2400, cache content that traverses the system 2400, store data about each of the devices in the system 2400 and perform any other typical functions of a database. In certain embodiments, the database 2455 may be connected to or reside within the communications network 2435, the communications network 2416, the communications network 2431, any other network, or a combination thereof In certain embodiments, the database 2455 may serve as a central repository for any information associated with any of the devices and information associated with the system 2400. Furthermore, the database 2455 may include a processor and memory or be connected to a processor and memory to perform the various operation associated with the database 2455. In certain embodiments, the database 2455 may be connected to the earphone devices 2415, 2430, the servers 2440, 2445, 2450, 2460, the first user device 2402, the second user device 2406, the third user device 2410, the fourth user device 2421, the fifth user device 2425, any devices in the system 2400, any other device, any network, or any combination thereof.

The database 2455 may also store information and metadata obtained from the system 2400, store metadata and other information associated with the first and second users 2401, 2420, store user profiles associated with the first and second users 2401, 2420, store device profiles associated with any device in the system 2400, store communications traversing the system 2400, store user preferences, store information associated with any device or signal in the system 2400, store information relating to patterns of usage relating to the first, second, third, fourth, and fifth user devices 2402, 2406, 2410, 2421, 2425, store audio content associated with the first, second, third, fourth, and fifth user devices 2402, 2406, 2410, 2421, 2425 and/or earphone devices 2415, 2430, store audio content and/or information associated with the audio content that is captured by the ambient sound microphones, store audio content and/or information associated with audio content that is captured by ear canal microphones, store any information obtained from any of the networks in the system 2400, store audio content and/or information associated with audio content that is outputted by ear canal receivers of the system 2400, store any information and/or signals transmitted and/or received by transceivers of the system 2400, store any device and/or capability specifications relating to the earphone devices 2415, 2430, store historical data associated with the first and second users 2401, 2415, store information relating to the size (e.g. depth, height, width, curvatures, etc.) and/or shape of the first and/or second user's 2401, 2420 ear canals and/or ears, store information identifying and or describing any eartip utilized with the earphone devices 2401, 2415, store device characteristics for any of the devices in the system 2400, store information relating to any devices associated with the first and second users 2401, 2420, store any information associated with the earphone devices 2415, 2430, store log on sequences and/or authentication information for accessing any of the devices of the system 2400, store information associated with the communications networks 2416, 2431, store any information generated and/or processed by the system 2400, store any of the information disclosed for any of the operations and functions disclosed for the system 2400 herewith, store any information traversing the system 2400, or any combination thereof. Furthermore, the database 2455 may be configured to process queries sent to it by any device in the system 2400.

The system 2400 may also include a software application, which may be configured to perform and support the operative functions of the system 2400, such as the operative functions of the first, second, third, fourth, and fifth user devices 2402, 2406, 2410, 2421, 2425 and/or the earphone devices 2415, 2430. In certain embodiments, the application may be a website, a mobile application, a software application, or a combination thereof, which may be made accessible to users utilizing one or more computing devices, such as the first, second, third, fourth, and fifth user devices 2402, 2406, 2410, 2421, 2425 and/or the earphone devices 2415, 2430. The application of the system 2400 may be accessible via an internet connection established with a browser program or other application executing on the first, second, third, fourth, and fifth user devices 2402, 2406, 2410, 2421, 2425 and/or the earphone devices 2415, 2430, a mobile application executing on the first, second, third, fourth, and fifth user devices 2402, 2406, 2410, 2421, 2425 and/or the earphone devices 2415, 2430, or through other suitable means. Additionally, the application may allow users and computing devices to create accounts with the application and sign-in to the created accounts with authenticating username and password log-in combinations. The application may include a custom graphical user interface that the first user 2401 or second user 2420 may interact with by utilizing a browser executing on the first, second, third, fourth, and fifth user devices 2402, 2406, 2410, 2421, 2425 and/or the earphone devices 2415, 2430. In certain embodiments, the software application may execute directly as an installed program on the first, second, third, fourth, and fifth user devices 2402, 2406, 2410, 2421, 2425 and/or the earphone devices 2415, 2430.

### Computing System for Facilitating the Operation and Functionality of the System

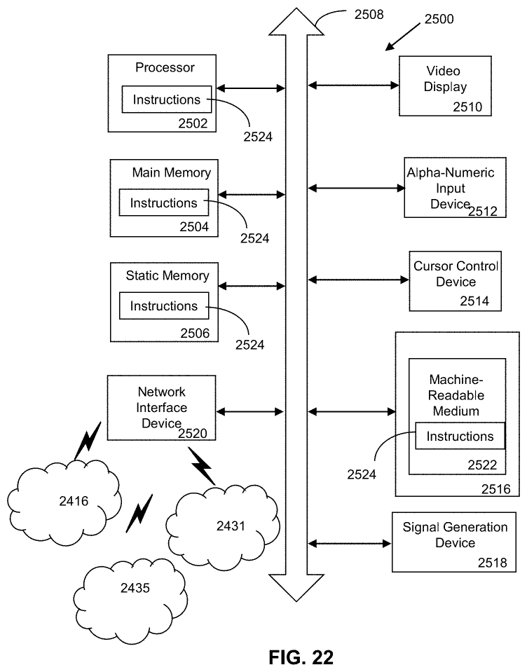

Referring now also to FIG. 22, at least a portion of the methodologies and techniques described with respect to the exemplary embodiments of the system 2400 can incorporate a machine, such as, but not limited to, computer system 2500, or other computing device within which a set of instructions, when executed, may cause the machine to perform any one or more of the methodologies or functions discussed above. The machine may be configured to facilitate various operations conducted by the system 2400. For example, the machine may be configured to, but is not limited to, assist the system 2400 by providing processing power to assist with processing loads experienced in the system 2400, by providing storage capacity for storing instructions or data traversing the system 2400, by providing functionality and/or programs for facilitating the operative functionality of the earphone devices 2415, 2430, and/or the first, second, third, fourth, and fifth user devices 2402, 2406, 2410, 2421, 2425 and/or the earphone devices 2415, 2430, by providing functionality and/or programs for facilitating operation of any of the components of the earphone devices 2415, 2430 (e.g. ear canal receivers, transceivers, ear canal microphones, ambient sound microphones, or by assisting with any other operations conducted by or within the system 2400.

In some embodiments, the machine may operate as a standalone device. In some embodiments, the machine may be connected (e.g., using communications network 2435, the communications network 2416, the communications network 2431, another network, or a combination thereof) to and assist with operations performed by other machines and systems, such as, but not limited to, the first user device 2402, the second user device 2411, the third user device 2410, the fourth user device 2421, the fifth user device 2425, the earphone device 2415, the earphone device 2430, the server 2440, the server 2450, the database 2455, the server 2460, or any combination thereof. The machine may be connected with any component in the system 2400. In a networked deployment, the machine may operate in the capacity of a server or a client user machine in a server-client user network environment, or as a peer machine in a peer-to-peer (or distributed) network environment. The machine may comprise a server computer, a client user computer, a personal computer (PC), a tablet PC, a laptop computer, a desktop computer, a control system, a network router, switch or bridge, or any machine capable of executing a set of instructions (sequential or otherwise) that specify actions to be taken by that machine. Further, while a single machine is illustrated, the term “machine” shall also be taken to include any collection of machines that individually or jointly execute a set (or multiple sets) of instructions to perform any one or more of the methodologies discussed herein.

The computer system 2500 may include a processor 2502 (e.g., a central processing unit (CPU), a graphics processing unit (GPU, or both), a main memory 2504 and a static memory 2506, which communicate with each other via a bus 2508. The computer system 2500 may further include a video display unit 2510, which may be, but is not limited to, a liquid crystal display (LCD), a flat panel, a solid state display, or a cathode ray tube (CRT). The computer system 2500 may include an input device 2512, such as, but not limited to, a keyboard, a cursor control device 2514, such as, but not limited to, a mouse, a disk drive unit 2516, a signal generation device 2518, such as, but not limited to, a speaker or remote control, and a network interface device 2520.

The disk drive unit 2516 may include a machine-readable medium 2522 on which is stored one or more sets of instructions 2524, such as, but not limited to, software embodying any one or more of the methodologies or functions described herein, including those methods illustrated above. The instructions 2524 may also reside, completely or at least partially, within the main memory 2504, the static memory 2506, or within the processor 2502, or a combination thereof, during execution thereof by the computer system 2500. The main memory 2504 and the processor 2502 also may constitute machine-readable media.

Dedicated hardware implementations including, but not limited to, application specific integrated circuits, programmable logic arrays and other hardware devices can likewise be constructed to implement the methods described herein. Applications that may include the apparatus and systems of various embodiments broadly include a variety of electronic and computer systems. Some embodiments implement functions in two or more specific interconnected hardware modules or devices with related control and data signals communicated between and through the modules, or as portions of an application-specific integrated circuit. Thus, the example system is applicable to software, firmware, and hardware implementations.

In accordance with various embodiments of the present disclosure, the methods described herein are intended for operation as software programs running on a computer processor. Furthermore, software implementations can include, but not limited to, distributed processing or component/object distributed processing, parallel processing, or virtual machine processing can also be constructed to implement the methods described herein.

The present disclosure contemplates a machine-readable medium 2522 containing instructions 2524 so that a device connected to the communications network 2435, the communications network 2416, the communications network 2431, another network, or a combination thereof, can send or receive voice, video or data, and communicate over the communications network 2435, the communications network 2416, the communications network 2431, another network, or a combination thereof, using the instructions. The instructions 2524 may further be transmitted or received over the communications network 2435, another network, or a combination thereof, via the network interface device 2520.

While the machine-readable medium 2522 is shown in an example embodiment to be a single medium, the term “machine-readable medium” should be taken to include a single medium or multiple media (e.g., a centralized or distributed database, and/or associated caches and servers) that store the one or more sets of instructions. The term “machine-readable medium” shall also be taken to include any medium that is capable of storing, encoding or carrying a set of instructions for execution by the machine and that causes the machine to perform any one or more of the methodologies of the present disclosure.

The terms “machine-readable medium,” “machine-readable device,” or “computer-readable device” shall accordingly be taken to include, but not be limited to: memory devices, solid-state memories such as a memory card or other package that houses one or more read-only (non-volatile) memories, random access memories, or other re-writable (volatile) memories; magneto-optical or optical medium such as a disk or tape; or other self-contained information archive or set of archives is considered a distribution medium equivalent to a tangible storage medium. The “machine-readable medium,” “machine-readable device,” or “computer-readable device” may be non-transitory, and, in certain embodiments, may not include a wave or signal per se. Accordingly, the disclosure is considered to include any one or more of a machine-readable medium or a distribution medium, as listed herein and including art-recognized equivalents and successor media, in which the software implementations herein are stored.

The illustrations of arrangements described herein are intended to provide a general understanding of the structure of various embodiments, and they are not intended to serve as a complete description of all the elements and features of apparatus and systems that might make use of the structures described herein. Other arrangements may be utilized and derived therefrom, such that structural and logical substitutions and changes may be made without departing from the scope of this disclosure. Figures are also merely representational and may not be drawn to scale. Certain proportions thereof may be exaggerated, while others may be minimized. Accordingly, the specification and drawings are to be regarded in an illustrative rather than a restrictive sense.

Thus, although specific arrangements have been illustrated and described herein, it should be appreciated that any arrangement calculated to achieve the same purpose may be substituted for the specific arrangement shown. This disclosure is intended to cover any and all adaptations or variations of various embodiments and arrangements of the invention. Combinations of the above arrangements, and other arrangements not specifically described herein, will be apparent to those of skill in the art upon reviewing the above description. Therefore, it is intended that the disclosure not be limited to the particular arrangement(s) disclosed as the best mode contemplated for carrying out this invention, but that the invention will include all embodiments and arrangements falling within the scope of the appended claims.

While the present invention has been described with reference to exemplary embodiments, it is to be understood that the invention is not limited to the disclosed exemplary embodiments. The scope of the following claims is to be accorded the broadest interpretation so as to encompass all modifications, equivalent structures and functions of the relevant exemplary embodiments. For example, if words such as “orthogonal”, “perpendicular” are used, the intended meaning is “substantially orthogonal” and “substantially perpendicular” respectively. Additionally, although specific numbers may be quoted in the claims, it is intended that a number close to the one stated is also within the intended scope, i.e. any stated number (e.g., 20 mils) should be interpreted to be “about” the value of the stated number (e.g., about 20 mils).

Thus, the description of the invention is merely exemplary in nature and, thus, variations that do not depart from the gist of the invention are intended to be within the scope of the exemplary embodiments of the present invention. Such variations are not to be regarded as a departure from the spirit and scope of the present invention.

Note that the durometer of the eartips can vary between 2 Shore A to 90 Shore A. Typical dimensions of the thickness of the membrane ending in the sealing section and lip or back ridge can be between approximately 0.001 mm to approximately 2 or more mm. The length (along the long axis) of an eartip can be from approximately 4 mm to approximately 25 mm or more depending upon the final usage. The outer diameter of contact portions of the eartip, such as sealing section 90, can vary from approximately 3 mm to approximately 50 or more mm, typically approximately 8 mm to approximately 18 mm. Note also that the thickness of the membrane of the body can be varied along the longitudinal length. For example the portion anticipated to contact the ear canal can be thinner, while the end of the membrane near the tip can be thicker to maintain restoring pressure.

The outer portion of an Eartip (e.g. a ridge) contacts the ear canal wall when inserted into the ear canal. The inner portion contains a core that can fit on a stent (earphone eartip), while a wider potion aids in insertion onto a stent, or if used as an earplug the core will be filled in. Prior to insertion into an ear canal the outer portion and inner portion encapsulate (Eartip membrane contacts a structure (e.g., stent part, ledge-movable or part of stent), when inserted or as presented (final form after folded from a negative mold)) a medium (e.g., gas, fluid) that can have an opening aiding molding. Note that the opening can be faced inward toward the ear canal or formed to face toward the ambient environment. Note that the stent can be fabricated from various materials (e.g., silicon, urethane, rubber) and can include internal

### Methods of Manufacturing

The Eartip (eartip) can be fabricated by various means, for example injection molding, then sealed with various filler mediums (e.g. gas, liquid, gel), and inserted upon a stent, for example the eartip can have an extension portion that slides over the stent.

For example specific materials may not be listed for achieving each of the targeted properties discussed, however one of ordinary skill would be able, without undo experimentation, to determine the materials needed given the enabling disclosure herein. For example Elastosil™ 30A, 70A, High Strength 1, 2, 3, Moldmaking Rubber (Alumilite™ products), flexible 3D printable material, silicon, urethane, natural and synthetic rubber, high strength rubber, chloroprene rubber, EVA rubber, quartz fiber, can be used; however, any material that can be used within the ear canal can be used for eartips and any material that can be used for earphones (silicon, urethane, rubber, plastic, Elastosil, metal, wood, and the like) can be used in the earphone housing and components thereof. As discussed herein, the eartips can be printed on three dimensional printers while provided the Shore A hardiness discussed herein. Various material can also be used for the EPH, for example tough resin (FormLabs) if printed and any other materials, as mentioned if molded. Typical durometer for the in ear portions can be from shore A of 5-50.

The eartips can be formed as an inverted shape mold as discussed herein. As an example, the inverted mold can be formed as a partial cylindrical sleeve with the shapes and arrangements disclosed herein. Such manufacturing provides great advantages of reduced cost without sacrificing performance of the eartips. As disclosed, the inverted shape mold allows the user to folder over portions of the eartip to use the eartip. Such eartips can provide the performance disclosed herein while being designed to be disposable.

The foregoing is provided for purposes of illustrating, explaining, and describing embodiments of this invention. Modifications and adaptations to these embodiments will be apparent to those skilled in the art and may be made without departing from the scope or spirit of this invention. Upon reviewing the aforementioned embodiments, it would be evident to an artisan with ordinary skill in the art that said embodiments can be modified, reduced, or enhanced without departing from the scope and spirit of the claims described below.

I claim:

1. An eartip comprising:
an outer portion; and
an inner portion, wherein the outer portion is designed to contact the ear canal, and wherein the inner portion is designed to fit upon a stent, where the eartip is formed by curing a combined mixture where the combined mixture is formed by combining 1-15% by weight or volume of lubricant with a remaining amount of elastomer.

2. The eartip according to claim 1, wherein the skin friction coefficient of the outer portion is less than 2.

3. The eartip according to claim 2, where the lubricant is a hydrophobic medium.

4. The eartip according to claim 3, where the hydrophobic medium is at least one of mineral oil, a wax, olive oil, and vegetable oil.

5. The eartip according to claim 4, where the combined mixture is formed by combining the hydrophobic medium by weight within the range of 3-8% of the weight of the combined mixture.

6. The eartip according to claim 4, where the combined mixture is formed by combining the hydrophobic medium by volume within the range of 3-8% of the volume of the combined mixture.

7. The eartip according to claim 5, where a hydrophobic coating is placed upon the outer portion.

8. The eartip according to claim 6, where a hydrophobic coating is placed upon the outer portion.

9. The eartip according to claim 8, where the hydrophobic coating is placed by dipping the ear tip outer surface into a treatment solution for period of time between 30 minutes to 3 hours.

10. The eartip according to claim 9, where the treatment solution is heated between 30 C to 150 C.

11. The eartip according to claim 5, where a hydrophobic coating is placed upon the outer portion.

12. The eartip according to claim 11, where the hydrophobic coating is placed by dipping the ear tip outer surface into a treatment solution for period of time between 30 minutes to 3 hours.

13. The eartip according to claim 12, where the treatment solution is heated between 30 C to 150 C.

14. A method for forming ear tips, the method comprising:
measuring one of a volume V1 of a lubricate or a weight W1 of a lubricant;
measuring one of a volume V2 of an elastomer epoxy or a weight W2 of an elastomer epoxy, where V1 or W1 is within a percentage range of 3 to 15% of V2 or W2;
mixing the lubricant with the epoxy to form a combined mixture during a work time of a curable epoxy;
insert the combined mixture into a mold used to form an eartip during the work time of the epoxy; and
removing the eartip from the mold after a cure time, where the eartip coefficient of friction is less than 2.0

15. The method according to claim 14, where the lubricant is a hydrophobic medium.

16. The eartip according to claim 15, where the hydrophobic medium is at least one of mineral oil, a wax, olive oil, and vegetable oil.

17. The eartip according to claim 16, where the combined mixture is formed by combining W1 with W2 where W1 is within the range of 3-8% of the total weight of W1+W2.

18. The eartip according to claim 16, where the combined mixture is formed by combining V1 with V2 where V1 is within the range of 3-8% of the total weight of V1+V2.
